(19) United States
(12) Patent Application Publication
(10) Pub. No.: US 2021/0386471 A1
(43) Pub. Date: Dec. 16, 2021

(54) ELECTROSURGICAL INSTRUMENT WITH IMPROVED SEALING

(71) Applicant: GYRUS MEDICAL LIMITED, Cardiff (GB)

(72) Inventor: Wilhelmus GOMMEREN, Cardiff (GB)

(73) Assignee: GYRUS MEDICAL LIMITED, Cardiff (GB)

(21) Appl. No.: 17/206,322

(22) Filed: Mar. 19, 2021

(30) Foreign Application Priority Data
Jun. 15, 2020 (GB) 2009050.2

(51) Int. Cl. A61B 18/14; A61B 18/12

(52) U.S. Cl. CPC A61B 18/14; A61B 2018/00077; A61B 18/1206

(57) ABSTRACT

An electrosurgical instrument is disclosed, having a first electrode having a planar face, a second electrode; an electrically insulative body, a fluid channel, a conductive wire, and a tubular seal. The tubular seal prevents saline from extending into a gap between the wire and the electrically insulative body of the instrument. The tubular seal is disposed around the wire, and extends between the wire and the body.

### FIELD OF THE INVENTION

The present invention relates to an electrosurgical instrument, and in particular to a radiofrequency (“RF”) electrosurgical instrument having at least one active electrode.

### BACKGROUND OF THE INVENTION

Electrosurgical instruments can be used for a variety of surgical methods. They may be used for tissue coagulation and sealing purposes, as well as tissue cutting. Electrosurgical end effectors can be combined with traditional surgical instruments in the same instrument.

A first example of a combined mechanical and electrosurgical instrument is described in U.S. Pat. No. 5,904,681, which describes an instrument having a mechanical cutting portion, such as a rotary burr, and an RF electrosurgical instrument which operates in a bipolar mode, for cutting and/or cauterizing. In this known instrument, a rotary burr removes hard tissues, such as bone, while the bipolar electrosurgical instrument is used to cut or ablate soft tissues and/or cauterize tissue, including blood vessels. In this known instrument, the mechanical cutting portion may include a rotary blade, which may be used for removing soft tissues, and the electrodes of the electrosurgical system may be used for cauterisation or coagulation. In order to provide RF electrical power to the electrosurgical instrument usually several wires run within the body of the instrument to connect the electrodes of the instrument to an RF electrosurgical generator. Effective sealing and insulation of these wires is required in order to prevent unwanted RF current leakage from the instrument, for example into a surrounding saline solution which might find its way into the instrument, which will adversely affect its operation.

### SUMMARY OF THE INVENTION

In order to prevent saline from leaking into a gap between the wire and an electrically insulative body in an electrosurgical instrument, the inventor has created a component that takes the form of a tubular seal. The tubular seal is disposed around the wire, and extends between an RF current providing wire and the insulative body. By using an additional component having a defined tubular shape, and providing the seal between the body and the wire, a fluid-tight interface between fluid at the surgical site and the wire is achieved. This prevents fluid, such as saline from the surgical site, leaking between the wire and the body, and so prevents current passing along an uncontrolled path from the wire through the leaked fluid to an electrode. An electrosurgical instrument having the seal of the present disclosure benefits from this advantage, while avoiding problems that the inventor has identified with using a glue, epoxy or resin. The tubular seal of the present disclosure is particularly advantageous in an electrosurgical instrument having an electrode with a substantially planar face attached to an electrically insulative body, due a gap around an edge of the planar face between the electrode and the body.

In view of the above, a first aspect of the invention provides an electrosurgical instrument having a tip end and a base end, the instrument comprising:
a first electrode having a substantially planar face;
a second electrode;
an electrically insulative body, disposed at least partly between the first and second electrodes, and at least partly surrounded on an outer edge by the second electrode;
a fluid channel disposed in the body, providing a fluid path that extends away from the tip end;
an electrically conductive wire, electrically connected to the first electrode, disposed in the electrically insulative body; and
an electrically insulative tubular seal, the seal being disposed around the electrically conductive wire at least partly between the wire and the body, so as to provide a fluid tight connection between the wire and the body.

The seal may extend along the wire up to the first electrode. This has the advantage of providing a reliable, robust, fluid tight connection. By positioning the seal such that it extends up to the first electrode, a robust assembly may be formed, in which the seal is secured in position. This also increases the overall area between the seal and the wire, reducing the likelihood of fluid passing along the inside of the seal around the wire.

The seal may have a tip end proximate the first electrode. The seal may have a base end. A diameter of the seal at the tip end may be larger than a diameter of the seal at the base end. This has the advantage of providing a reliable, robust fluid-tight connection. In this arrangement, a compressive force on the seal from the body may be larger at the tip end of the seal, closer to the first electrode and to the fluid channel opening, and at a location that prevents fluid from reaching the remainder of the wire within the body. This difference in diameter also provides an arrangement which is easy to assemble.

The seal may be tapered. This may be such that the diameter of the seal gradually decreases away from its tip end towards its base end. This has the advantage of providing an electrosurgical instrument which is easy to manufacture and assemble, as the taper can act as a guide to assembly.

The seal may comprise a radial extension at its tip end. The radial extension may be aligned with the body and/or the first electrode. This has the advantage of providing an electrosurgical instrument which is easy to manufacture and assemble. The radial extension may provide a guide during assembly, by providing a ledge of the seal to be aligned with the body. The radial extension also increases the overall area between the seal and the body, reducing the likelihood of fluid passing around the seal into the body, and into contact with the wire.

The seal may comprise an elastically deformable material, such as an elastomeric material. This has the advantage of providing a reliable connection between the seal and the body, which has displacement tolerance. Specifically, the wire may be displaced radially by a fraction, and any such displacement may be accommodated by the seal with the fluid-tight connection being maintained. This also has the advantage of providing an instrument that is easy to manufacture and assemble.

The seal may comprise a bioinert material. This has the advantage of providing a seal and instrument that is configured for use in a surgical environment.

The seal may have a wall thickness in a radial direction. The wall thickness may be larger at a tip end of the seal than at a base end of the seal. This has the advantage of providing an electrosurgical instrument which is easy to manufacture and assemble. This also has the advantage of permitting a dimension change in the diameter of the seal, while being compatible with a wire of uniform diameter.

A second aspect of the invention provides a method of assembling an electrosurgical instrument, comprising:
providing a first electrode, a second electrode, an electrically insulative body, an electrically conductive wire, and a tubular seal, as defined in any of the embodiments or combinations provided herein,
inserting the wire and seal simultaneously into the electrically insulative body, and
securing the first and second electrodes to the electrically insulative body.

Inserting the wire and seal simultaneously into the electrically insulative body may be carried out from a tip end of the electrosurgical instrument. This may be by feeding the wire and seal towards a base end of the electrosurgical instrument.

An electrosurgical system may be provided, comprising:
the electrosurgical instrument as defined in any of the embodiments or combinations provided herein; and
an electrical power source.

The electrosurgical system may further comprise a fluid suction source.

There may be provided a method of using the device. The method may involve performing surgery on a human or animal patient using the electrosurgical instrument. The method may include one or more of the steps of: providing an electrical current to the first electrode; providing suction from the fluid channel; using the first electrode in a surgical step.

### BRIEF DESCRIPTION OF THE DRAWINGS

Embodiments of the invention will now be described with reference to the accompanying drawings, in which:

FIG. 1 is a section perspective view of a prior art electrosurgical instrument;

FIG. 2 is a cross-section of the prior art electrosurgical instrument of FIG. 1;

FIG. 3 is a schematic illustrating an electrosurgical system;

FIG. 4 is a cross-section of an electrosurgical instrument according to the present disclosure; and

FIG. 5 is a cross-section of an embodiment of a seal of the present disclosure.

### DETAILED DESCRIPTION OF EMBODIMENT(S)

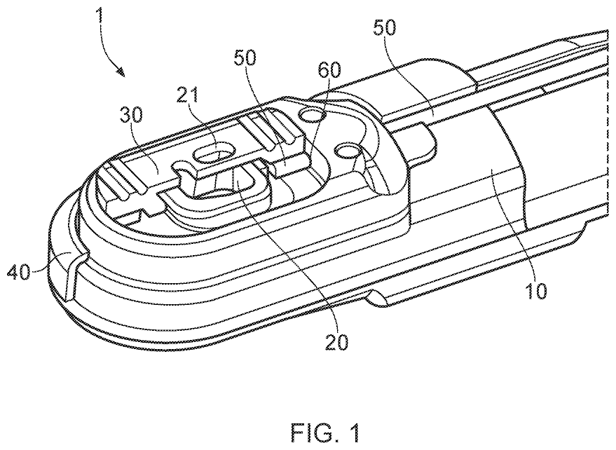

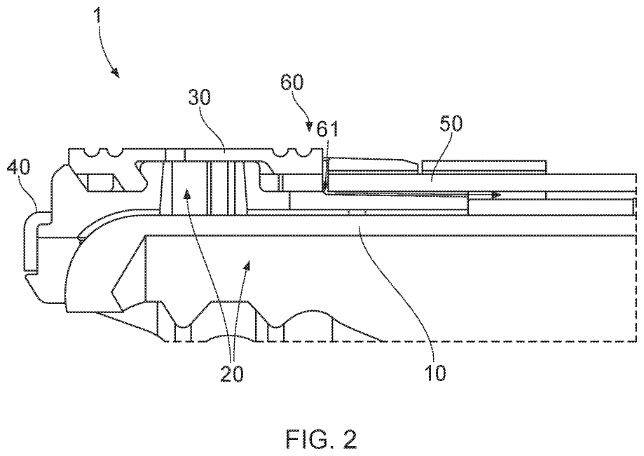

An example electrosurgical instrument 1 is shown in FIGS. 1 and 2. This electrosurgical instrument comprises a body 10, a fluid channel 20, a fluid channel opening 21, a first electrode 30, a second electrode 40, and a conductive wire 50. As a skilled person in the field will appreciate, the body 10 is electrically insulative and the electrodes 30, 40 and conductive wire 50 are electrically conductive. The electrosurgical instrument is of the type known as a rotary shaver, which combines a rotary mechanical cutting blade with RF electrosurgical electrodes to provide for tissue ablation or sealing.

In a surgical method involving this electrosurgical instrument 1, the surgical site is provided with saline solution. The fluid channel 20 and fluid channel opening 21 are fluidly connected to a suction source (not shown), so that saline solution and biological material at the surgical site can be removed through the fluid channel opening 21, through the fluid channel 20, towards the suction source.

The inventor of the present invention has identified that in the electrosurgical instrument 1 shown in FIGS. 1 and 2 and described above, fluid at the site of operation can leak into the electrosurgical instrument 1, through a gap 60, to the wire 50, along a path 61 between the first electrode 30 and the body 10, and between the wire 50 and the body 10. This may cause the first electrode 30 to be fired up at the wrong location. To mitigate this problem, a resin, glue or epoxy (for example, under the brand name EPO-TEK® can be inserted into the gap 60, at the location identified in FIG. 1 by reference numeral 60. By using resin or glue here, the interface between the first electrode 30 and the body 10 is sealed, so fluid cannot pass further into the device. However, the inventor has identified that this is not an ideal solution. Glue takes time to cure, which can increase assembly time, and increases costs involved in the assembly process. Glue or resin is also difficult to control, as variables such as temperature, pressure, chemical composition of the surrounding atmosphere, and amount of glue can affect curing time and extent, causing a problem with lack of uniformity between the final products, which in turn causes problems with quality control reliability, and cost. Glue or resin can also reduce the aesthetic appearance of the electrosurgical instrument.

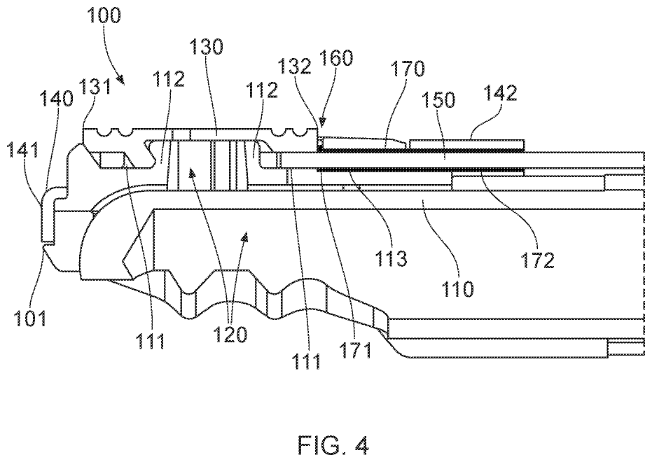

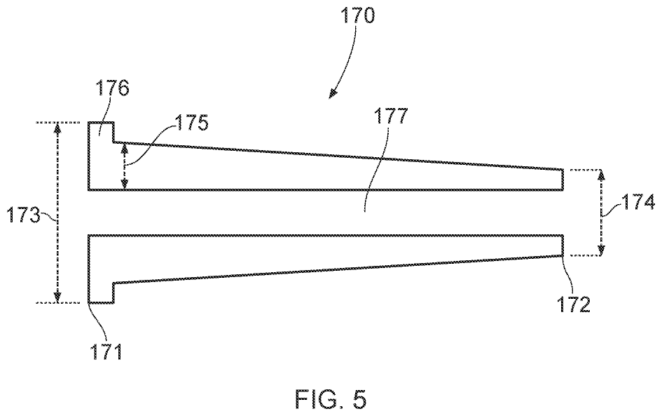

In order to address the above, and as explained previously in the Summary of the Invention, and with reference to FIG. 4, the electrosurgical instrument 100 described herein has a tubular seal 170 that prevents saline from extending into a gap between the wire 150 and the electrically insulative body 110 of the instrument 100. The tubular seal 170 is disposed around the wire 150, and extends between the wire 150 and the body 110. By using a seal 170 having a defined tubular shape, and providing the seal 170 between the body 110 and the wire 150, a fluid-tight interface is achieved. This prevents fluid, such as saline from the surgical site, leaking between the wire 150 and the body 110, and so prevents current passing along an uncontrolled path from the wire 150 through the leaked fluid to an electrode 130. An embodiment of a tubular seal 170 is shown in FIG. 5.

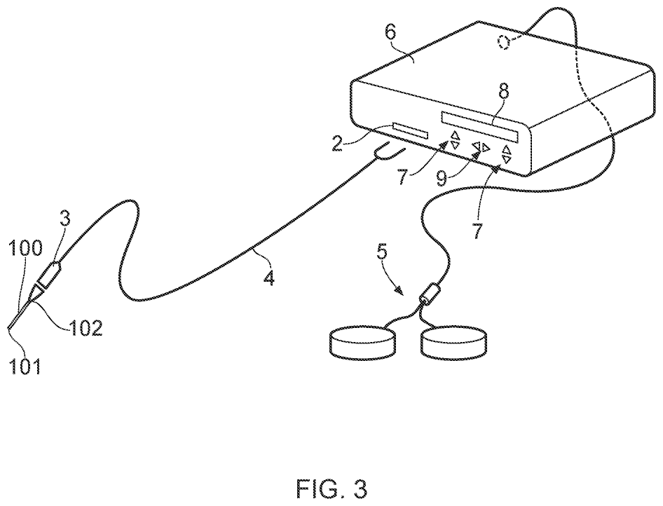

The electrosurgical instrument 100 described herein may be provided as part of an electrosurgical system. An embodiment of an electrosurgical system is shown in FIG. 3. The system may comprise an electrosurgical instrument 100 having a handle portion 3, which may be connected to an RF output from an output socket 2 of an electrosurgical generator 6, via a connection cord 4. The instrument 100 may have irrigation and/or suction tubes (not shown) which may be connected to an irrigation fluid and/or fluid suction source (not shown). The generator 6 may comprise an electrical power source (not shown). Activation of the generator 6 may be performed from the instrument 100 via a handheld switch (not shown) on the instrument 100, or by means of a foot-switch unit 5. The foot-switch unit may be provided with two switches for selecting different operational modes of the generator 6 and instrument 100, such as a coagulation mode or a cutting or vaporisation (ablation) mode. Push buttons 9 may be provided as an alternative means for selection between the ablation (cutting) and coagulation modes. The generator 6 may comprise push buttons 7 for setting ablation and/or coagulation power levels, which may be indicated in a display 8.

An embodiment of the electrosurgical instrument 100 is shown in FIG. 4, having a tip end 101 and a base end 102 (shown in FIG. 3). The electrosurgical instrument 100 comprises a first electrode 130, a second electrode 140, an electrically insulative body 110, a fluid channel 120, an electrically conductive wire 150, and an electrically insulative tubular seal 170. The electrosurgical instrument 100 is configured so that application of a current through the first electrode 130 can apply an electrical current to the surgical site, and may be used in a surgical operation, which may involve burning human or animal biological tissue.

The first electrode 130 has a substantially planar face. The first electrode 130 may be an active electrode. The first electrode 130 may comprise or consist of an electrically conductive material, or have a conductive coating or conductive surface layer. The first electrode 130 may comprise a fluid channel opening, which is fluidly connected to the fluid channel 120. The first electrode 130 may have a tip edge 131 and a base edge 132.

The second electrode 140 may be a return electrode, and may be configured to direct current away from the surgical site, towards a generator 6. The second electrode 140 may extend around the electrically insulative body 110. The second electrode 140 may extend perimetrically or in the case of an insulative body 110 having a substantially circular cross-section, circumferentially, around the insulative body 110, and/or may extend around the tip end 101 of the insulative body 110. The second electrode 140 may have a first band 141 that extends around the tip 101 of the insulative body 110, and/or may have a second band 142 that extends perimetrically around the insulative body 110.

The insulative body 110 may be substantially elongate. The electrically insulative body 110 may comprise or consist of an electrically insulative material. The insulative body 110 may comprise or consist of ceramic, including but not limited to one or more of: alumina, zirconia toughened alumina (ZTA), and yttria stabilized zirconia (YTZP). The insulative body 110 may comprise or consist of a plastic material, including but not limited to a thermoplastic polymer such as a polyether ether ketone (PEEK).

The insulative body 110 is disposed at least partly between the first and second electrodes 130, 140. The insulative body 110 may define an opening 113 configured to accommodate at least part of the wire 150. Part of the wire 150 and the tubular seal 170 may be disposed in the opening 113.

The insulative body 110 is at least partly surrounded on an outer edge by the second electrode 140. The second electrode 140 may define an opening, and a portion of the insulative body 110 may extend from the opening. The opening may be defined by a first band 141 of the second electrode 140. The portion of the electrically insulative body 110 itself may define an opening 111. The first electrode 130 may be disposed in the opening of the insulative body 110. The insulative body 110 may comprise an extension 112 within the opening, such that the opening is substantially annular and disposed around the extension 112. The first electrode 130 may be configured to clip onto the extension 112. The first electrode 130 may be configured to cover and/or surround the extension 112.

The first electrode 130 may be disposed in the opening 111 of the insulative body 110 such that the substantially flat, planar face of the first electrode 130 is substantially aligned with an outer surface of the insulative body 110. The first electrode 130 and/or insulative body 110 may be sized or dimensioned so as to provide a gap 160 between the first electrode 130 and insulative body 110 when assembled to one another. The gap 160 may be provided between a second edge 132 of the first electrode 130 and the insulative body 110.

The fluid channel 120 is disposed in the insulative body 110, and provides a fluid path that extends away from the tip end 101 of the instrument. The fluid channel 120 may provide a path that extends from the first electrode 130, and specifically from an opening in the first electrode 130, towards a centre of the insulative body 110, then along a centre of the insulative body 110 away from the tip end 101. Where the insulative body 110 comprises an extension 112, the fluid channel 120 may extend through the extension 112. The fluid channel 120 may be a fluid removal channel. The fluid channel 120 may be configured for removal of saline, tissue, any other fluid at the surgical site, or any other debris at the surgical site. The fluid channel 120 may be fluidly connected to a suction source, such as a vacuum source.

The electrically conductive wire 150 is electrically connected to the first electrode 130, and is disposed in the electrically insulative body 110. The wire 150 may be directly connected to the first electrode 130. The wire 150 may be disposed proximate and/or directly connected at or proximate a base edge 132 of the first electrode 130. The wire 150 may extend away from a base edge 132 of the first electrode 131 towards a base end 102 of the instrument. The wire 150 may be elongate. The wire 150 may be substantially cylindrical, although the skilled person will appreciate that any appropriate shape of wire may be used, such as a ribbon-shaped wire, and in particular a substantially flat ribbon-shaped wire. The wire 150 may have a substantially uniform diameter along its length, or have a varying diameter along its length. The wire 150 may comprise or consist of a metal or alloy.

The electrically insulative tubular seal 170 is disposed around the electrically conductive wire 150 at least partly between the wire 150 and the body 110, so as to provide a fluid tight seal between the wire 150 and the body 110. As shown in FIG. 4, the seal 170 may extend along the wire 150 up to the first electrode 130. As a skilled person will appreciate, the term tubular is not limited to only a cylindrical tubular shape, and may include, for example, a ribbon-shape, and in particular a substantially flat ribbon shape. In order to accommodate a ribbon-shaped wire 150, the seal 170 may have a substantially ribbon-shaped internal surface. Additionally or alternatively, the seal 170 may have a substantially ribbon-shaped external surface. In such a case, the seal 170 may have a substantially rectangular cross-section, defined by either or both of its internal and external surfaces.

The seal 170 may have a tip end 171, which may be proximate the first electrode 130, specifically proximate a base edge 132 of the first electrode 130, and a base end 172, and a diameter of the seal 170 at the tip end 171 may be larger than a diameter of the seal 170 at the base end 172. The seal 170 may be substantially cylindrical. The seal 170 may comprise a radial extension 176 (which may be as illustrated in FIG. 5) at its tip end 171, the radial extension 176 being aligned with the body 110 and/or with the first electrode 130. The radial extension 176 may have a width 173 in a radial direction. The seal base end 172 may have a width 174. The seal base end width 174 may be smaller than the radial extension width 173. The radial extension 176 may be a ledge or a bung. The radial extension 176 may be substantially circular, and may be substantially disc-shaped. The radial extension 176 may be aligned with a radius of the seal 170, such that it defines a plane intersected at a normal by a longitudinal axis of the seal 170.

The seal 170 may be tapered, such that at least part of the seal 170 has a diameter that gradually decreases away from its tip end 171 towards its base end 172. The seal 170 may have a tapered portion, connected to the radial extension 176. The tapered portion may extend away from the radial extension 176, such that its width decreases gradually away from the radial extension 176. The seal 170 may have a wall thickness 175 in a radial direction, and the wall thickness 175 may be larger at a tip end 171 of the seal 170 than at a base end 172 of the seal 170. Alternatively or in addition, the wire 150 may have an increased thickness proximate the tip end 171 of the seal 170. The wire 150 may be tapered away from this increased thickness, towards and/or away from the tip end 101 of the instrument.

The seal 170 may define an internal bore 177, configured to accommodate the wire 150. The internal bore 177 may be disposed substantially centrally within the seal 170, and may be aligned with/extend along a longitudinal axis of the seal 170. The internal bore 177 may be substantially elongate and/or cylindrical. The seal 170 may be fixedly attached and, optionally, bonded to at least part of the wire 150. This bonding may be along an entire surface of the bore 177. Alternatively, the seal 170 may be fixedly attached to the wire by means of an interference fit, and optionally by an elastic compressive force provided by the seal 170. The seal 170 may be a wire sheath or sleeve. The seal 170 may be attached to the wire 150 such that the wire extends from the tip end 171 and/or the base end 172 of the seal 170. A longer part of the wire 150 may extend from the base end 172 than from the tip end 171. The wire 150 may be configured relative to the seal 170 such that a length of wire extending from the tip end 171 extends up to the first electrode 130. The seal 170 may be fixedly attached and, optionally, bonded to at least part of an electrically insulative sheath of the wire 150. The seal 170 may be provided as a single component with an insulative sheath of the wire 150, and optionally as a single unitary piece of material with an electrically insulative sheath of the wire 150.

The seal 170 may be a sealing component, and may be a single unitary piece of material. The seal 170 may comprise or consist of an elastically deformable material, such as an elastomeric material. Alternatively, the seal 170 may be substantially rigid. The seal 170 may comprise or consist of a bioinert material. The seal 170 may have a bioinert or biocompatible coating. The seal 170 may comprise or consist of a polymeric material such as one or more of: poly(methyl methacrylate); poly(ethylene); polyethylene glycol; polyurethane; polysiloxane; polyamide. The seal 170 may comprise or consist of an electrically insulating material.

The seal 170 may be visually identifiable or have a microstructure indicative of having been made by injection moulding, having been cast, or having been extruded.

The electrosurgical instrument 100 may be assembled using a method comprising the following steps:
providing a first electrode 130, a second electrode 140, an electrically insulative body 110, an electrically conductive wire 150, and a tubular seal 170, as defined in any embodiment described herein,
inserting the wire 150 and seal 170 simultaneously into the electrically insulative body 110, and
securing the first and second electrodes 130, 140 to the electrically insulative body 110.

The method may further include any, all, or any appropriate combination of the steps:
securing the wire 150 and seal 170 to one another;
securing the wire 150 to the first electrode 130;
securing the seal 170 to the electrically insulative body 110.

The wire 150 may be attached to the first electrode 130, and the wire 150 and first electrode 130 may be assembled to the insulative body 140 in a single step.

The wire 150 and seal 170 may be inserted into the insulative body 110 such that a base end 172 of seal 170 is inserted into the body 110 before any other part of the seal 170. The seal 170 may be inserted into the insulative body 110 until a radial extension 176 of the seal 170 contacts the body 110. The wire 150 and seal 170 may be inserted into the body 110 by applying a force to a tip end 171 of the seal 170. Any force applied to insert the seal 170 and wire 150 into the insulative body 110 may be removed at the point at which a radial extension 176 of the seal 170 contacts the insulative body 110.

The steps outlined above may be performed in the order listed, performed simultaneously, or in any appropriate order. For example, inserting the wire 150 and seal 170 into the electrically insulative body 110 may be carried out simultaneously. This may be carried out from a tip end 101 of the electrosurgical instrument 100, by feeding the wire 150 and seal 170 towards a base end 102 of the electrosurgical instrument 100.

Although the invention has been described above with reference to one or more preferred embodiments, it will be appreciated that various changes or modifications may be made without departing from the scope of the invention as defined in the appended claims.

Any reference numerals used in the claims should be construed as a guide to a possible embodiment or embodiments only, and not be construed as limiting on the scope of the claims.

1. An electrosurgical instrument having a tip end and a base end, the instrument comprising:
a first electrode having a substantially planar face;
a second electrode;
an electrically insulative body, disposed at least partly between the first and second electrodes, and at least partly surrounded on an outer edge by the second electrode;
a fluid channel disposed in the body, providing a fluid path that extends away from the tip end;
an electrically conductive wire, electrically connected to the first electrode, disposed in the electrically insulative body; and
an electrically insulative tubular seal, the seal being disposed around the electrically conductive wire at least partly between the wire and the body, so as to provide a fluid tight seal between the wire and the body.

2. The electrosurgical instrument of claim 1, wherein the seal extends along the wire up to the first electrode.

3. The electrosurgical instrument of claim 1, wherein the seal has a tip end proximate the first electrode, and a base end, and a diameter of the seal at the tip end is larger than a diameter of the seal at the base end.

4. The electrosurgical instrument of claim 3, wherein the seal is tapered, such that its diameter gradually decreases away from its tip end towards its base end.

5. The electrosurgical instrument of claim 1, wherein the seal comprises a radial extension at its tip end, the radial extension being aligned with the body and/or the first electrode.

6. The electrosurgical instrument of claim 1, wherein the seal comprises an elastically deformable material.

7. The electrosurgical instrument of claim 1, wherein the seal comprises a bioinert material.

8. The electrosurgical instrument of claim 1, wherein the seal has a wall thickness in a radial direction, and the wall thickness is larger at a tip end of the seal than at a base end of the seal.

9. A method of assembling an electrosurgical instrument, comprising:
providing a first electrode, a second electrode, an electrically insulative body, an electrically conductive wire, and a tubular seal, as defined in claim 1,
inserting the wire and seal simultaneously into the electrically insulative body, and
securing the first and second electrodes to the electrically insulative body.

10. The method of claim 9, wherein inserting the wire and seal simultaneously into the electrically insulative body is carried out from a tip end of the electrosurgical instrument, by feeding the wire and seal towards a base end of the electrosurgical instrument.

11. An electrosurgical system, comprising:
an electrosurgical instrument having a tip end and a base end, the instrument comprising: a first electrode having a substantially planar face; a second electrode; an electrically insulative body, disposed at least partly between the first and second electrodes, and at least partly surrounded on an outer edge by the second electrode; a fluid channel disposed in the body, providing a fluid path that extends away from the tip end; an electrically conductive wire, electrically connected to the first electrode, disposed in the electrically insulative body; and an electrically insulative tubular seal, the seal being disposed around the electrically conductive wire at least partly between the wire and the body, so as to provide a fluid tight seal between the wire and the body; the system further comprising:
an electrosurgical generator, the electrosurgical instrument being operatively coupled to the generator when in use to receive RF electrosurgical signals therefrom.

12. The electrosurgical system of claim 11, further comprising a fluid suction source.
